(19) United States
(12) Patent Application Publication
(10) Pub. No.: US 2009/0124642 A1
(43) Pub. Date: May 14, 2009

(54) Crystalline forms of Erlotinib HCI and formulations thereof

(76) Inventors: Augusto Canavesi, Locate Varesino (IT); Marco Villa, Milano (IT); Ales Gavenda, Ostrava - Lhotka (CZ); Jiri Faustmann, Opava (CZ); Judith Aronhime, Rehovot (IL); Ettore Gibatti, Rho (IT); Alexandr Jegorov, Dobra Voda (CZ)

Correspondence Address: MERCHANT & GOULD PC, P.O. BOX 2903, MINNEAPOLIS, MN, 55402-0903, US

(21) Appl. No.: 12/229,682

(22) Filed: Aug. 25, 2008

Related U.S. Application Data
(60) Provisional application No. 60/957,585, filed on Aug. 23, 2007.
(60) Provisional application No. 60/984,348, filed on Oct. 31, 2007.
(60) Provisional application No. 61/052,943, filed on May 13, 2008.
(60) Provisional application No. 61/073,990, filed on Jun. 19, 2008.
(60) Provisional application No. 61/018,160, filed on Dec. 31, 2007.
(60) Provisional application No. 61/128,658, filed on May 22, 2008.
(60) Provisional application No. 61/082,671, filed on Jul. 22, 2008.
(60) Provisional application No. 60/990,813, filed on Nov. 28, 2007.
(60) Provisional application No. 61/059,204, filed on Jun. 5, 2008.
(60) Provisional application No. 61/075,174, filed on Jun. 24, 2008.

(51) Int. Cl. A61K 31/517; C07D 239/94

(52) U.S. Cl. 514/266.4; 544/293

(57) ABSTRACT

The invention provides a novel crystalline form of Erlotinib HCl, processes for its preparation, and formulations thereof.

### CROSS-REFERENCE TO RELATED APPLICATIONS

This application claims the benefit of U.S. provisional application Ser. Nos. 60/957,585, filed Aug. 23, 2007; 60/984,348, filed Oct. 31, 2007; 61/052,943, filed May 13, 2008; 61/073,990, filed Jun. 19, 2008; 60/968,207, filed Aug. 27, 2007; 61/018,160, filed Dec. 31, 2007; 61/128,658, filed May 22, 2008; 61/082,671, filed Jul. 22, 2008; 60/990,813, Nov. 28, 2007; 61/059,204, Jun. 5, 2008 and 61/075,174, filed Jun. 24, 2008, each of which is incorporated herein by reference.

### FIELD OF THE INVENTION

The invention relates to crystalline forms of Erlotinib HCl, polymorphic pure crystalline form of Erlotinib HCl, preparation thereof and formulation thereof.

### BACKGROUND OF THE INVENTION

Erlotinib (ERL) HCl, N-(3-ethynylphenyl)-6,7-bis(2-methoxyethoxy)-4-quinazolinamine hydrochloride, of the following formula

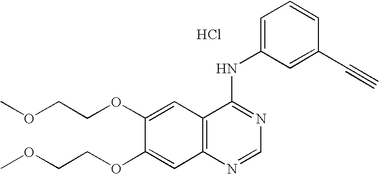

is marketed under the trade name TARCEVA® by OSI pharmaceuticals for treatment of patients with locally advanced or metastatic non-small cell lung cancer (NSCLC) after failure of at least one prior chemotherapy regimen.

Erlotinib and its preparation are disclosed in U.S. Pat. No. 5,747,498; where the free base is produced, as shown in Scheme 1, by reaction of 3-ethynylaniline (3-EBA) with 4-chloro-6,7-bis(2-methoxyethoxy)quinazoline (CMEQ) in a mixture of pyridine and isoproanol (EPA). The free base is isolated and purified by chromatography on silica gel using a mixture of acetone and hexane. The base is converted into the hydrochloride by treating a solution of Erlotinib in CHCl₃/Et₂O with HCl.

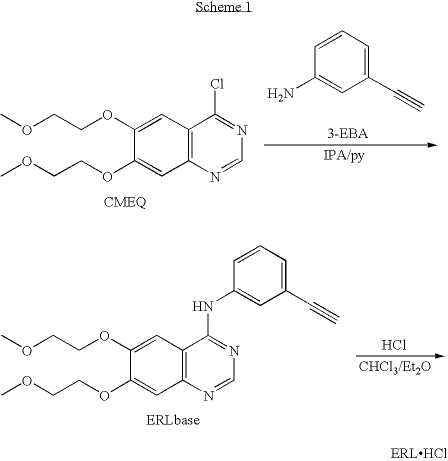

U.S. Pat. No. 6,900,221 discloses Form A that exhibits an X-ray powder diffraction pattern having characteristics peaks expressed in degrees 2-theta at 5.579, 6.165, 7.522, 8.006, 8.696, 9.841, 11.251, 19.517, 21.152, 21.320, 22.360, 22.703, 23.502, 24.175, 24.594, 25.398, 26.173, 26.572, 27.080, 29.240, 30.007, 30.673, 32.759, 34.440, 36.154, 37.404 and 38.905; and Form B substantially free of Form A, wherein Form B exhibits an X-ray powder diffraction pattern having characteristics peaks expressed in degrees 2-theta at approximately 6.255, 7.860, 9.553, 11.414, 12.483, 13.385, 14.781, 15.720, 16.959, 17.668, 17.193, 18.749, 19.379, 20.196, 20.734, 21.103, 21.873, 22.452, 22.982, 23.589, 23.906, 24.459, 25.138, 25.617, 25.908, 26.527, 26.911, 27.534, 28.148, 28.617, 29.000, 29.797, 30.267, 30.900, 31.475, 31.815, 32.652, 33,245, 34.719, 35.737, 36.288, 36.809, 37.269, 37.643 and 38.114.

This patent also reports pharmaceutical compositions of Form A and of the pure Form B, wherein Form B is present in the composition in an amount of at least 70% by weight as compared to the amount of Form A. The patent also relates to a method for producing crystalline Erlotinib HCl which according to it should be more suitable for tables and oral administration, and consists essentially of the pure Form B, which is considered by them to be more stable thermodynamically.

U.S. Pat. No. 6,900,221 also states that “the hydrochloride compound disclosed in U.S. Pat. No. 5,574,498 actually comprised a mixture of the polymorphs A and B, which because of its partially reduced stability (i.e., from the polymorph A component) was not more preferred for tablet form than the mesylate forms.”

U.S. Pat. No. 6,476,040 discloses methods for the production and crystallizations of Erlotinib and salts, the production is done by treatment of 4-[3-[[6,7-bis(2-methoxyethoxy]-4-quinazolinyl]amino]phenyl]-2-methyl-3-butyn-2-ol with sodium hydroxide and then with HCl in IPA, 2-methoxyethanol, 2-butanol and n-butanol) as reported in Scheme 2.

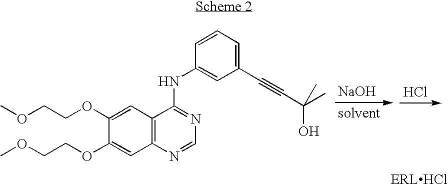

U.S. Pat. No. 7,148,231 discloses Forms A, B, E, which are characterized by X-Ray powder diffraction, IR and melting point.

The present invention relates to the solid state physical properties of Erlotinib HCl. These properties can be influenced by controlling the conditions under which Erlotinib HCl is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take this fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance that can be identified unequivocally by X-ray spectroscopy. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by solid state ¹³C NMR spectrometry and infrared spectroscopy.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

### SUMMARY OF THE INVENTION

One embodiment of the invention provides crystalline Erlotinib HCl characterized by data selected from the group consisting of: a powder XRD pattern with peaks at about 5.9, 9.7, 11.7, 16.2, 21.7 and 23.3±0.2 degrees two-theta; a PXRD pattern depicted in FIG. 1; a PXRD pattern depicted in FIG. 2; a solid-state ¹³C NMR spectrum with signals at about 150.0, 136.1, 134.3 and 126.8±0.2 ppm; a solid-state ¹³C NMR spectrum having chemical shift differences between the signal exhibiting the lowest chemical shift and another in the chemical shift range of 100 to 180 ppm of about 48.4, 34.4, 32.6 and 25.2±0.1 ppm; a solid-state ¹³C NMR spectrum depicted in FIG. 4; a solid-state ¹³C NMR spectrum depicted in FIG. 5, and combination thereof.

One embodiment, the invention encompasses crystalline Erlotinib HCl characterized by data selected from the group consisting of: a powder XRD pattern having peaks at about 9.7, 11.2, and 21.1±0.2 degrees two-theta, and at least any 3 peaks selected from the list consisting of 5.6, 16.9, 24.0, 25.3 and 26.0±0.2 degrees 2-theta; a PXRD pattern depicted in FIG. 7; a PXRD pattern depicted in FIG. 8; a solid-state ¹³C NMR spectrum with signals at about 155.4, 148.6, 138.1, 129.4 and 102.3±0.2 ppm; a solid-state ¹³C NMR spectrum depicted in FIG. 10; and a solid-state ¹³C NMR spectrum depicted in FIG. 11, and combination thereof.

Yet another embodiment of the invention provides a formulation comprising at least one of the above crystalline forms of Erlotinib HCl and at least one pharmaceutically acceptable excipient.

One embodiment of the invention provides a pharmaceutical composition comprising at least one of the above crystalline forms of Erlotinib HCl prepared according to the processes of the present invention, and at least one pharmaceutically acceptable excipient.

Another embodiment of the invention provides a process for preparing a pharmaceutical formulation comprising combining at least one of the above crystalline forms of Erlotinib HCl with at least one pharmaceutically acceptable excipient.

Yet another embodiment of the invention provides a process for preparing a pharmaceutical composition comprising at least one of the above crystalline forms of Erlotinib HCl, prepared according to the processes of the present invention, and at least one pharmaceutically acceptable excipient.

One embodiment of the invention provides the use of the above crystalline forms of Erlotinib HCl of the present invention for the manufacture of a pharmaceutical composition.

### BRIEF DESCRIPTION OF THE FIGURES

FIG. 1 illustrates a powder X-ray diffraction pattern of crystalline form G of Erlotinib HCl (in a polymorphic pure state).

FIG. 2 illustrates a zoomed-in powder X-ray diffraction pattern of crystalline form G of Erlotinib HCl (in a polymorphic pure state).

FIG. 3 illustrates a DSC thermogram of crystalline form G of Erlotinib HCl (in a polymorphic pure state).

FIG. 4 illustrates a solid-state ¹³C-NMR spectrum of crystalline form G of Erlotinib HCl (in a polymorphic pure state).

FIG. 5 illustrates a solid-state ¹³C-NMR spectrum in the range of 190-100 ppm of crystalline form G of Erlotinib HCl (in a polymorphic pure state).

FIG. 6 Microscope figure of crystalline form G of Erlotinib hydrochloride.

FIG. 7 illustrates a powder X-ray diffraction pattern of crystalline form F of Erlotinib HCl.

FIG. 8 illustrates a zoom-in powder X-ray diffraction pattern of crystalline form F of Erlotinib HCl.

FIG. 9: illustrates a DSC thermogram of crystalline form F of Erlotinib HCl.

FIG. 10: illustrates a solid-state ¹³C-NMR spectrum of crystalline form F of Erlotinib HCl.

FIG. 11: illustrates a solid-state ¹³C-NMR spectrum in the range of 190-100 ppm of crystalline form F of Erlotinib HCl.

FIG. 12: illustrates a Microscope figure of crystalline form F of Erlotinib hydrochloride

### DETAILED DESCRIPTION OF THE INVENTION

As used herein, the term “room temperature” refers to a temperature of about 18° C. to about 30° C., preferably about 19° C. to about 28° C. and more preferably about 20° C. to about 25° C.

As used herein, unless otherwise defined, the term “Form A” when referring to crystalline erlotinib hydrochloride means a crystalline form of erlotinib hydrochloride that exhibits an X-ray powder diffraction pattern having characteristic peaks expressed in degrees 2-theta at approximately 5.7, 9.8, 10.1, 10.3, 18.9, 19.5, 21.3, 24.2, 26.2 and 29.2±0.2 degrees 2-theta.

As used herein, unless otherwise defined, the term “Form B,” when referring to crystalline erlotinib hydrochloride means a crystalline form of erlotinib hydrochloride that exhibits an X-ray powder diffraction pattern having characteristic peaks expressed in degrees 2-theta at approximately 6.3, 7.8, 9.5, 12.5, 13.4, 20.2, 21.1, 22.4 and 28.9±0.2 degrees 2-theta.

The present invention relates to a crystalline forms of erlotinib hydrochloride, polymorphic pure crystalline forms of erlotinib hydrochloride, methods for preparation thereof, and pharmaceutical formulation, comprising the same.

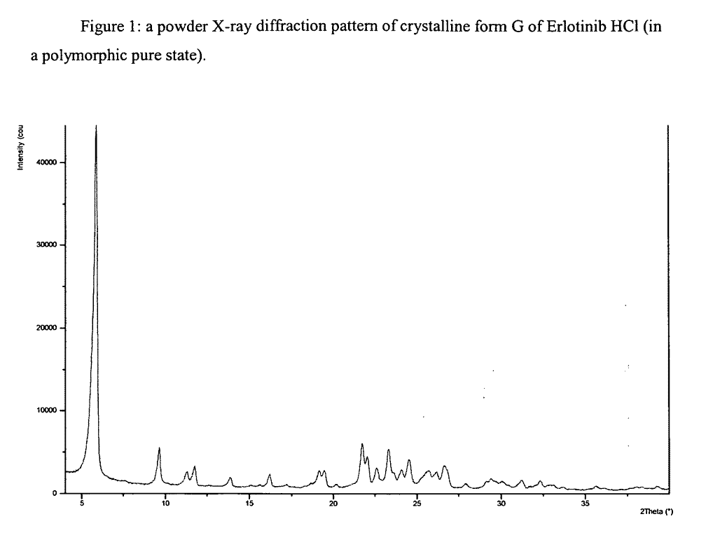

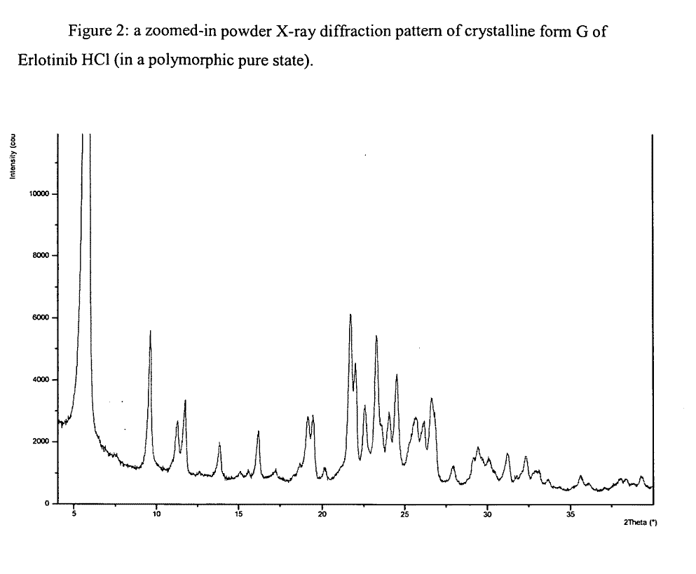

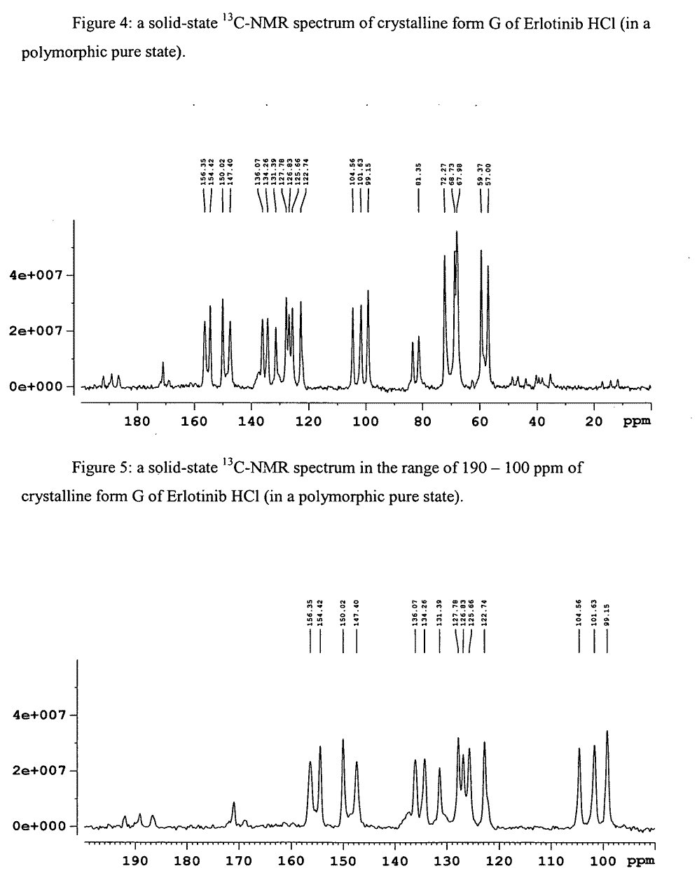

One embodiment of the invention provides crystalline form G of Erlotinib HCl, characterized by data selected from the group consisting of: a powder XRD pattern with two fixed peaks at about 5.9, 11.7 and 2-3 peaks selected from a group of peaks at about 9.7, 11.3, 13.9 and 19.1±0.2 degrees two-theta; a powder XRD pattern with peaks at about 5.9, 9.7, 11.7, 16.2, 21.7 and 23.3±0.2 degrees two-theta; a PXRD pattern depicted in FIG. 1; a PXRD pattern depicted in FIG. 2; a solid-state ¹³C NMR spectrum with signals at about 150.0, 136.1, 134.3 and 126.8±0.2 ppm; a solid-state ¹³C NMR spectrum having chemical shift differences between the signal exhibiting the lowest chemical shift and another in the chemical shift range of 100 to 180 ppm of about 48.4, 34.4, 32.6 and 25.2±0.1 ppm; a solid-state ¹³C NMR spectrum depicted in FIG. 4; and a solid-state ¹³C NMR spectrum depicted in FIG. 5, and combination thereof. Typically, the signal exhibiting the lowest chemical shift in the chemical shift range of 100 to 180 ppm is at about 101.6±1 ppm.

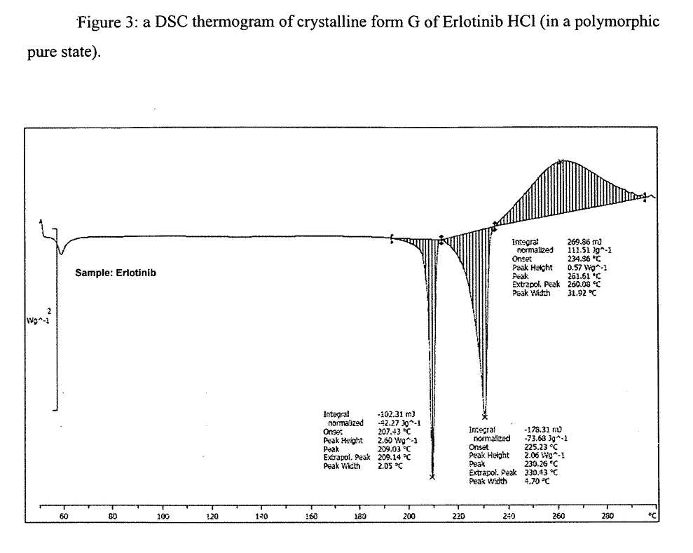

The crystalline Form G Erlotinib HCl of the present invention can be further characterized by data selected from the group consisting of: a DSC thermogram having peaks at about 209° C. and 230° C.; a DSC thermogram depicted in FIG. 3; a powder XRD pattern with peaks at about 11.3, 13.9, 19.1, 19.5, 22.5 and 24.5±0.2 degrees two-theta, and a solid-state ¹³C NMR spectrum with signals at about 156.4, 154.4, 147.4 and 131.4±0.2 ppm.

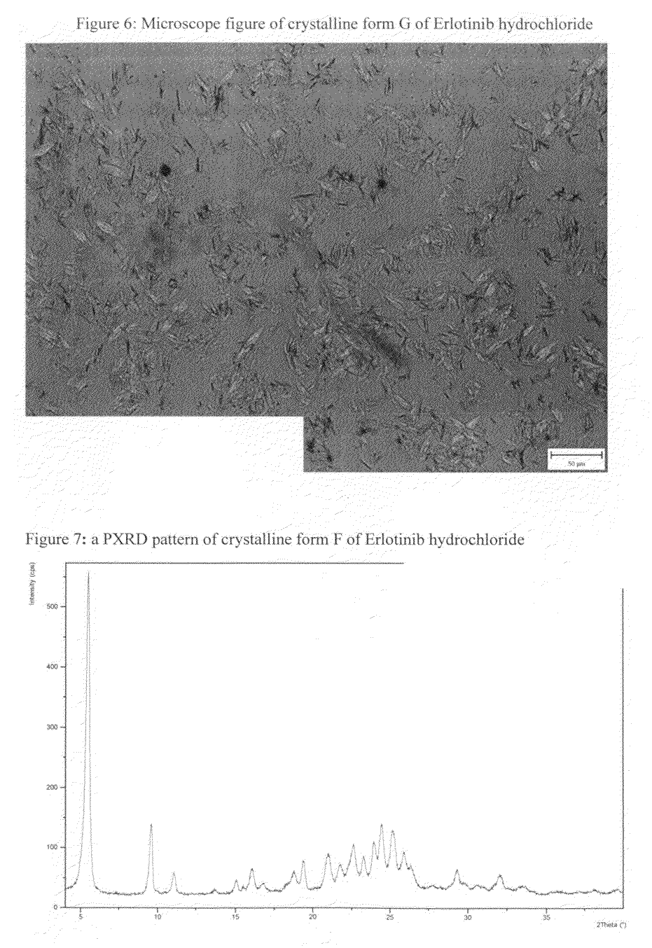

The crystalline form G of the present invention can be also characterized by laurel leaf-like particle shape (particles are flat shaped) as depicted in FIG. 6.

The above polymorph is provided in a polymorphic pure state. As used herein, unless mentioned otherwise, the term “polymorphic pure”, in reference to the above crystalline form G of ERL-HCl means crystalline Erlotinib HCl containing no more than about 15% by weight of crystalline Erlotinib HCl Form A or B, preferably not more than 10% by weight of Form A or B, more preferably not more than 5% by weight of Form A or B. Typically, the content of Form A in the crystalline form G of Erlotinib HCl is measured by PXRD or by ¹³C solid state NMR.

Typically, the amount of form A in the crystalline form G of ERL-HCl of the present invention is measured by PXRD using any peak from the group of peaks at about: 5.7, 9.8, 10.1, 10.3, 18.9, 22.8 and 24.3 deg±0.2 degrees 2-theta and the amount of form B in the said form is measured by PXRD using any peak from group of peaks at about: 6.2, 7.8, 12.5, 13.4, 16.9 and 21.1 deg±0.2 degrees 2-theta. Typically, the amount of form A in the crystalline form G of ERL-HCl of the present invention is measured by ¹³C solid state NMR using any peak from group of peaks at about: 172.0, 149.7, 137.4, 130.5 and 122.1±0.2 ppm and the amount of form B in the said form is measured by ¹³C solid state NMR using any peak from group of peaks at about: 158.2, 108.5 and 106.0±0.2 ppm.

The above crystalline form G of erlotinib HCl is an anhydrous form of Erlotinib hydrochloride. As use herein, unless mentioned otherwise, the term “anhydrous” in reference to the crystalline Erlotinib HCl of the present invention means a substance having a weight loss not more than about 1% by TGA, more preferably, not more than about 0.5% by TGA.

The above crystalline form G is prepared by a process comprising reacting erlotinib base with HCl in dry 1,3-dioxalane, butanol or mixtures thereof, providing a suspension comprising the said crystalline Form G of erlotinib HCl, and recovering the above crystalline Erlotinib HCl. Preferably, the recovered crystalline ERL-HCl is polymorphic pure. Preferably, HCl is added to the solution no more than 1 hour after the formation of the solution, more preferably, as soon as it is formed, i.e., without delay.

Preferably, the process comprises dissolving dry Erlotinib base in dry 1,3-dioxalane, butanol or mixtures thereof; and admixing the solution with HCl, providing a suspension comprising the crystalline Form G Erlotinib HCl of the present invention.

As used herein, the term “dry” in reference to Erlotinib and 1,3-dioxalane means a substance having a water content of less than about 0.1% by weight, preferably, less than 0.09% by weight.

Preferably, dissolution of erlotinib base in the said solvents is done at about room temperature to about 80° C., depending on the solvent. Preferably, when the solvent is butanol, the dissolution is done at about room temperature to about 80° C., more preferably, at about 80° C. Preferably, when the solvent is dry 1,3-dioxalane, the dissolution is done at a temperature of about room temperature to about 74° C.-75° C., more preferably, at about room temperature.

Erlotinib base can be prepared, for example, according to the process disclosed in U.S. Pat. No. 5,747,498, example 20.

Typically, admixing the solution of Erlotinib base and HCl is an exothermic reaction, thus the mixing can be done at low temperatures. Preferably, HCl is added to the solution of erlotinib base in a dry 1,3-dioxalane, or mixture of butanol and a small amount of water. Preferably, the addition is done at a temperature of about 0° C. to about 70° C.

Preferably, HCl is provided in a form of a concentrated solution. Preferably, the solvent of the HCl solution is diethylether, butanol or mixtures thereof. Preferably, the concentration of the solution is about 5 to about 19% by weight/volume, more preferably, about 19% by weight/volume. Typically, such HCl solution is prepared by bubbling HCl gas into diethylether, butanol or mixtures thereof. Determination of the concentration of the HCl solution is done by titrations with a base, as known to one skilled in the art.

Typically, the addition of HCl to the solution provides a suspension comprising of a precipitate of the crystalline Erlotinib HCl of the present invention.

The suspension can be further maintained. Preferably, the suspension is maintained for about 15 minutes to about 1 hour. Preferably, the suspension is maintained at a temperature of about 0° C. to about 30° C.

The suspension can be further cooled and maintained. Preferably, the suspension is further cooled to a temperature of about −5° to about +5° C., more preferably to about 0° C. Preferably, the cooled suspension is further maintained for about 24 hours.

The recovery of the crystalline Erlotinib HCl of the present invention from the suspension can be done for example by filtering and drying.

Preferably, drying is done at a temperature of about 40° C. to about 60° C., preferably, for a period of about 1 hour to about overnight.

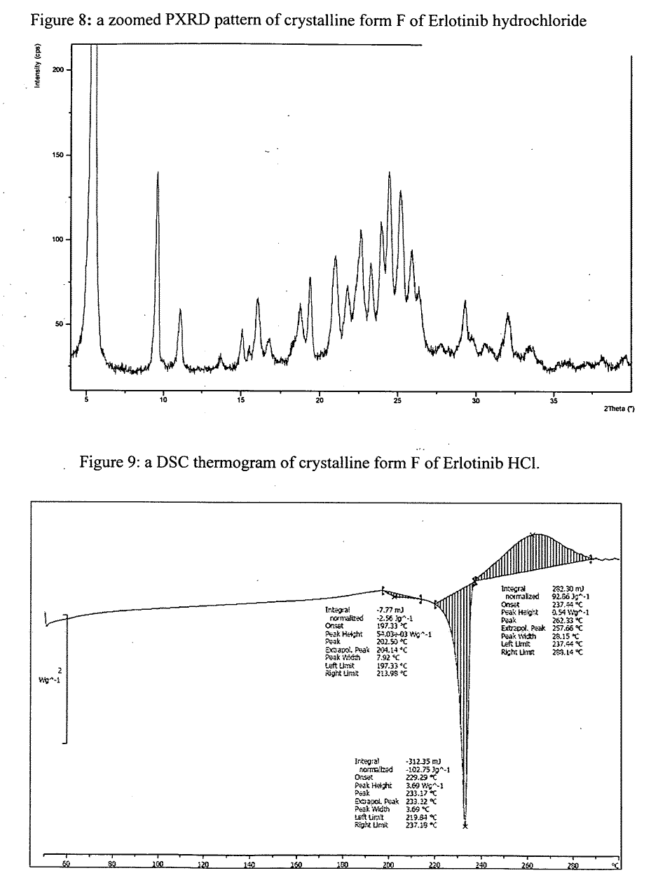

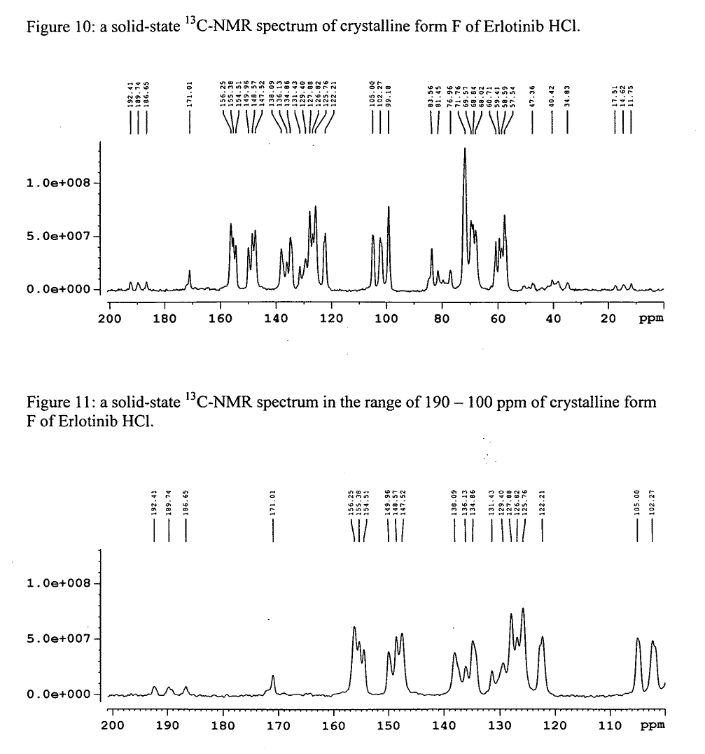

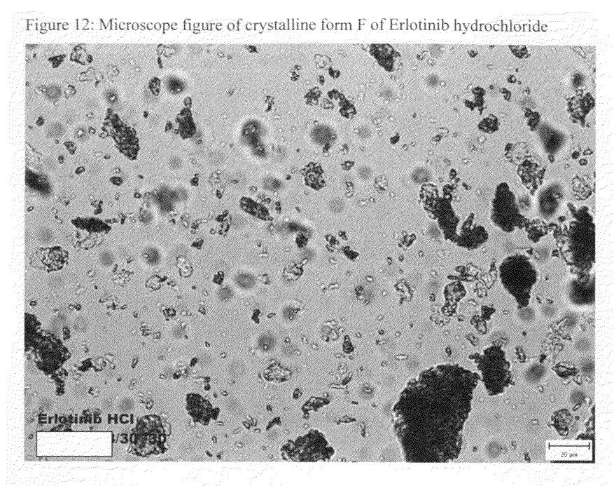

The present invention encompasses crystalline form F of Erlotinib HCl, characterized by data selected from the group consisting of: a powder XRD pattern having peaks at about 9.7, 11.2, and 21.1±0.2 degrees two-theta, and at least any 3 peaks selected from the list consisting of 5.6, 16.9, 24.0, 25.3 and 26.0±0.2 degrees 2-theta; a PXRD pattern depicted in FIG. 7; a PXRD pattern depicted in FIG. 8; a solid-state ¹³C NMR spectrum with signals at about 155.4, 148.6, 138.1, 129.4 and 102.3±0.2 ppm; a solid-state ¹³C NMR spectrum depicted in FIG. 10; and a solid-state ¹³C NMR spectrum depicted in FIG. 11, and combination thereof.

The crystalline Form F Erlotinib HCl of the present invention can be further characterized by data selected from the group consisting of: a DSC thermogram having peaks at about 203° C. and 233° C.; a DSC thermogram depicted in FIG. 9.

The above second crystalline form can be prepared by a process comprising:

a) dissolving Erl-base in dioxolane,

b) maintaining the solution for more than an hour prior to the addition of HCl, and

c) adding HCl to obtain a suspension comprising the said crystalline form

Preferably, dioxalan contains about 0.031% by weight of water.

Preferably, prior to maintaining the solution it is heated. Preferably, the solution is heated to about 20° C. to about 70° C., more preferably, about 30° C. to about 60° C.

Preferably, the solution in step b is maintained upon stirring. Preferably, the solution is maintained for more than 1 hour.

Preferably, the concentration of the HCl added is about 44.1% w/v. Preferably, the molar ratio between Erlotinib and HCl is about 1:1.

Preferably, HCl is added while stirring. Preferably, the stirring speed is about 700 rpm to about 1100 rpm.

Preferably, after HCl addition the suspension is maintained for a period of about 5 minutes to about 10 minutes. Preferably the suspension is maintained at temperature of about 20° C. to about 70° C., more preferably, at about 30° C. to about 60° C.

The process for preparing the second crystalline form may further comprise cooling the suspension prior to recovering the said crystalline form. Typically, during the cooling granulation occurs. Preferably, cooling is done to a temperature of about 0° C. Preferably, granulating is preformed for about 30 minutes to about 60 minutes.

The process for preparing the second crystalline form may further comprise recovery of the said crystalling form from the suspension. Preferably, the said recovery comprises:

a) separation of said precipitated solid, and

b) drying the said separated solid

Preferably, the solid is separated by filtration.

Preferably, the drying is done by a stream of N₂ gas. Preferably, said drying is done at a temperature of about 60° C., preferably for a period of about 1 hour to about 20 hours.

The above polymorph is provided in a polymorphic pure state. As used herein, unless mentioned otherwise, the term “polymorphic pure”, in reference to the above crystalline form F of ERL-HCl means crystalline Erlotinib HCl containing no more than about 15% by weight of crystalline Erlotinib HCl Forms A, B or G, preferably not more than 10% by weight, most preferably not more than 5% by weight. Typically, the content of other form in crystalline form F Erlotinib HCl of the present invention is measured by PXRD or by ¹³C solid state NMR. Typically, the amount of form A in the crystalline form F of ERL-HCl is measured by PXRD using any peak from the group of peaks at about: 9.8, 10.1, 10.3 and 11.4±0.2 degrees 2-theta. Typically, the amount of form B in the crystalline form F of ERL-HCl is measured by PXRD using any peak from the group of peaks at about: 6.2, 7.8, 12.5, 13.4 and 20.1±0.2 degrees 2-theta. Typically, the amount of crystalline form G of the present invention in crystalline form F of ERL-HCl of the present invention is measured by PXRD using any peak from the group of peaks at about: 5.9, 11.7 and 19.1±0.2 degrees 2-theta.

The above crystalline Forms of Erlotinib HCl of the present invention can then be used for the manufacture of a pharmaceutical composition. Thus, the invention provides formulation and process for making thereof comprising of at least one of the crystalline Forms of Erlotinib HCl and at least one pharmaceutically acceptable excipient. Preferably, the crystalline ERL-HCl of the present invention that are used for the formulation are polymorphic pure. Preferably, the pharmaceutical composition is packed in a form of a tablet.

Direct compression, however, is generally limited to those circumstances in which the active ingredient has physical characteristics suitable for forming pharmaceutically acceptable tablets. These physical characteristics include, but are not limited to, good flowing properties, compressibility, and compactability.

Direct compression formulations comprising the pure crystalline of Erlotinib HCl of the present invention is developed, because the crystals of the pure crystalline Erlotinib HCl of the present invention are suitable for direct compression formulations.

The method for making tablets by direct compression comprises providing a mixture of pure crystalline Erlotininb HCl of the present invention, at least one diluent, at least one tablet binder, and at least one tablet disintegrant; blending the mixture to obtain a homogeneous mixture; adding at least one tablet lubricant to the homogeneous mixture; and compressing the homogeneous mixture in a tablet press to obtain tablets. Optionally, at least one colorant may be added to the mixture to provide any desired colored tablet.

Diluents used in the mixture include diluents commonly used for tablet preparation. For example, diluents include, but are not limited to, calcium carbonate, calcium phosphate (dibasic and/or tribasic), calcium sulfate, powdered cellulose, dextrates, dextrin, fructose, kaolin, lactitol, anhydrous lactose, lactose monohydrate, maltose, mannitol, microcrystalline cellulose, sorbitol, sucrose, or starch. Preferably, the diluent is lactose monohydrate, microcrystalline cellulose, or starch. Typically, the diluent is present in an amount of about 35 to about 85 percent by weight of the tablet. Preferably, the diluent is present in an amount of about 40 to about 80 percent by weight of the tablet. Preferably, the amount of diluent relative to the amount of Erlotinib hydrochloride is about 50-70% of diluent.

Binders are agents used to impart cohesive qualities to the powdered material. Binders impart cohesiveness to the tablet formulation that ensures that the tablet remains intact after compression. Tablet binders used in the mixture include tablet binders commonly used for tablet preparation. Tablet binders include, but are not limited to, acacia, alginic acid, carbomer, sodium carboxymethylcellulose, dextrin, ethylcellulose, gelatin, glucose, guar gum, hydroxypropyl cellulose, maltose, methylcellulose, polyethylene oxide, or povidone. Preferably, the tablet binder is hydroxypropyl cellulose. Typically, the tablet binder is present in an amount of about 0.5 to about 5 percent by weight of the tablet. Preferably, the tablet binder is present in an amount of about 0.7 to about 3 percent by weight of the tablet.

A disintegrant is a substance or mixture of substances added to a tablet formulation to facilitate a tablet's breakup or disintegration after tablet administration. The Erlotinib HCl should be released from the tablet as efficiently as possible to allow dissolution. Tablet disintegrants used in the mixture include, but are not limited to, at least one of alginic acid, sodium croscarmellose, crospovidone, maltose, microcrystalline cellulose, potassium polacrilin, sodium starch glycolate, or starch. Preferably, the tablet disintegrant is a “super-disintegrant:”crospovidone, sodium starch glycolate or sodium croscarmellose. Typically, the tablet disintegrant is present in an amount of about 3 to about 15 percent by weight of the tablet. Preferably, the tablet disintegrant is present in an amount of about 5 to about 10 percent by weight of the tablet.

The blending step is carried out to substantially homogeneous mixture. The skilled artisan with little or no experimentation can easily determine the equipment and conditions necessary for the blending steps. Factors that may influence the blending step include, but are not limited to, the amount of materials, the physical characteristics of the materials, the equipment, and the speed of mixing.

Lubricants have a number of functions in tablet manufacturing. For example, lubricants prevent adhesion of the tablet material to equipment, reduce interparticle friction, and facilitate the ejection of the tablet from the die cavity, among others. Tablet lubricants added to the homogeneous mixture include those typically used in tablet formulations. Tablet lubricants include, but are not limited to, at least one of calcium stearate, glyceryl behenate, magnesium stearate, mineral oil, polyethylene glycol, sodium stearyl fumarate, stearic acid, talc, or zinc stearate. Preferably, the tablet lubricant is magnesium stearate. Typically, the tablet lubricant is present in an amount of about 0.5 to about 2 percent by weight of the tablet. Preferably, the tablet lubricant is present in an amount of about 0.7 to about 1 percent by weight of the tablet.

The compressing step may be carried out using a tablet compression apparatus commonly used in tableting. For example, a Kilian tableting press may be used to form the tablets.

Once a tablet is made using the methodology described above, the pure crystalline form of Erlotinib HCl of the present invention in the tablet can be detected by the techniques known by the skilled in the art, especially powder X-Ray diffraction or solid-state NMR (of carbon or nitrogen).

The invention also encompasses tablets made using the methodology described above. In one embodiment the tablet comprises the pure crystalline forms of Erlotinib HCl of the present invention, lactose monohydrate, microcrystalline cellulose, magnesium stearate, hydroxyptopylmethyl cellulose and sodium dodecylsulphate.

Having described the invention with reference to certain preferred embodiments, other embodiments will become apparent to one skilled in the art from consideration of the specification. The invention is further defined by reference to the following examples describing in detail the formation of dry compression pharmaceutical formulations of pure crystalline form of Erlotinib HCl of the present invention and the dissolution of the tablets made using the dry compression pharmaceutical formulations. It will be apparent to those skilled in the art that many modifications, both to materials and methods, may be practiced without departing from the scope of the invention.

### EXAMPLES

### PXRD

XRPD diffraction was performed on X-Ray powder diffractometer: PanAlytical X'pert Pro powder diffractometer, Cu-tube, scanning parameters: CuKa radiation, λ=1.541874 Å; equipped with X'celerator detector, active length 2.122 mm Scanning parameters: Range:4-40 degrees two-theta; Continuous scan; 6 deg./min; Sample holder: a round standard stainless steel sample holder with round zero background silicon plate with cavity. Prior to analysis the samples were gently ground by means of mortar and pestle in order to obtain a fine powder. The ground sample was adjusted into a cavity of the sample holder and the surface of the sample was smoothed by means of a microscopic glass slide.

### DSC

DSC measurements were performed on Differential Scanning Calorimeter DSC823e (Mettler Toledo). Al crucibles 40 μl with PIN were used for sample preparation.

Typical weight of sample was 1-3 mg.
Program: temperature range 50° C.-300° C., 10° C./min.

### TGA

TGA measurements were performed on instrument TGA/SDTA 851e (Mettler Toledo). Alumina crucibles 70 μl were used for sample preparation. Usual weight of sample was 8-12 mg. Program: temperature range 25° C.-250° C., 10° C./min.

### Solid-State NMR

Bruker Avance 500 WB/US NMR spectrometer (Karlsruhe, Germany, 2003). 125 MHz, Magic angle spinning (MAS) frequency 11 kHz, 4 mm ZrO2 rotors and standard CPMAS pulse program was used.

### Crystal 16

The Crystal16 (manufactured by Avantium Technologies) is a multiple reactor station designed for carrying out crystallization studies at a 1 ml scale.

### Microscope

An optical microscope system with polarized light, CCD camera and data software.

### Example 1

### Preparation of Pure Crystalline Form G of Erlotinib Hydrochloride of the Present Invention

Erlotinib base (waterless, 500 mg, 1.271 mmole) was dissolved in dry 1,3-dioxolane (20 ml). The temperature of the solution was adjusted at 0° C. and 112.2 μl (mole/mole) of concentrated hydrochloric acid (concentration of 41% w/v was determined by acidobasic titration) was added to the solution of Erlotinib base. Solid phase was created immediately. The crystalline suspension was agitated for 1 hr at 0° C. and then let to stay overnight in a refrigerator (0° C.). Then the crystalline phase was separated by filtration, rinsed with 1,3-dioxolane (10 ml) and dried on the filter by blowing nitrogen through the cake to the constant weight. The drying was finished in a small laboratory oven under a flow of nitrogen at 60° C. for 4 hrs.

Pure crystalline Erlotinib hydrochloride was obtained (506 mg, yield 92.6%).

### Example 2

### Preparation of Pure Crystalline Form G of Erlotinib Hydrochloride of the Present Invention

Erlotinib base (waterless, 50 mg, 0.1271 mmole) was dissolved in dry 1,3-dioxolane (2 ml). Temperature of the solution was adjusted at 30° C. and 45.9 μl (mole/mole) of 10.1% w/v HCl in ether was added to the solution of Erlotinib base. Solid phase was created immediately. The crystalline suspension was agitated for 1 hr at 30° C. and then cooled to 0° C. The crystalline phase was separated by filtration and dried in small laboratory oven under nitrogen ventilation at 40° C. for 3 hrs. Pure crystalline Erlotinib hydrochloride was obtained (46.2 mg, yield 84.6%).

### Example 3

### Preparation of a Dry Pharmaceutical Formulation of Pure Crystalline Form G and Crystalline Form F of Erlotinib Hydrochloride of the Present Invention

A crystalline G of erlotinib hydrochloride, having the main PXRD peaks at 5.9, 9.7, 11.3, 11.7, 13.8, 23.3 and 24.6±0.2 degrees two-theta, and or crystalline Form F of erlotinib hydrochloride of the present invention, having the main PXRD peaks at 5.6, 9.7, 11.2, 16.9, 24.0 and 26.0±0.2 degrees two-theta, and all the components presented in the below table were weighed together and mixed to obtain a tablet. Components for formulation were weighed in the quantity as mentioned in table bellow or in the corresponding ratio.

| | | |
|---|---|---|
| Erlotinib hydrochloride | 111 | mg |
| Lactose monohydrate | 103 | mg |
| Magnesium stearate | 3 | mg |
| Microcrystalline celulose | 49 | mg |
| Hydroxypropylmethyl celulose | 49 | mg |
| Sodium dodecylsulphate | 10 | mg |
| Total weight | 325 | mg |

Then, the tablet was pressed and analyzed by PXRD. The analysis of formulation of crystalline form G of the present invention providing the following main PXRD peaks at 5.9, 9.7, 11.3, 11.7, 13.8, 23.3 and 24.6±0.2, which belong to pure crystalline form G of Erlotinib hydrochloride of the present invention. The analysis of formulation of crystalline form F of the present invention providing the following main PXRD peaks at 5.6, 9.7, 11.2, 16.9, 24.0 and 26.0±0.2, which belong to crystalline Form F of Erlotinib hydrochloride of the present invention.

### Example 4

### Preparation of Pure Crystalline Form G of Erlotinib Hydrochloride of the Present Invention

2 g of Erlotinib base was dissolved at 80° in 20 g of butanol, a solution of 0.5 g 32% aqueous HCl in butanol was added and suspension was cooled at room temperature, the crystals were filtered after 15 minutes, rinsed with butanol and dried at 50° under vacuum overnight. 1.8 g of product was obtained.

### Example 5

### Preparation of the Crystalline Form G of Erlotinib Hydrochloride of the Present Invention

0.50 g of Erlotinib base+20 ml of 1,3-dioxalane (water content: 0.031%) were placed into a magnetic stirred bulb and the temperature inside was adjusted at +30° C. Immediately after dissolution of base 105 Oil of concentrated hydrochloric acid (44.1% w/v HCl; 1 eq) was added via an electronic burette, maintaining the stirring speed at 700 rpm. A crystalline solid was appeared immediately after the addition. The temperature was kept at 30° C. for additional 10 minutes. Then the suspension was cooled down and after about 30 minutes of granulation at 0° C. the solid was separated on a filter and dried at 60° C./1 hrs/N2. Crystalline form E (0.50 g; molar yield 91.5%) was obtained.

### Example 6

### Preparation of Crystalline Form G of Erlotinib Hydrochloride of the Present Invention

28.6 mg of Erlotinib base+1.15 ml of 1,3-dioxalane (water content: 0.031%) were placed into a magnetic stirred glass-vial. After that the temperature inside was adjusted at +30° C. which resulted in dissolution of the base. The precipitation was performed immediately after dissolution of the base, which take several minutes. 6.0 μl of concentrated hydrochloric acid (44.1% w/v HCl; 1 eq) was added by a microsyringe, maintaining the stirring speed at 1100 rpm. A crystalline solid was appeared immediately. The temperature was kept at 30° C. for additional 10 minutes. After that the suspension was cooled down and after about 30 minutes of granulation at 0° C. the solid was separated on a filter and dried at 60° C./1 hrs/N2. Crystalline form E (22.0 mg; molar yield 70.4%) was obtained.

### Example 7

### Preparation of Crystalline Form F of Erlotinib Hydrochloride in Crystal16

Precipitation in Crystal16: 25 mg of Erl-base+1 ml of dioxolane (0.031% water) were dissolved. After that the temperature+30° C. was adjusted and the solution was stirred for the duration of one hour; HCl (44.1% w/v; 5.25 l; molar ratio approximately 1:1) was added by a microsyringe, maintaining the stirring speed at 1100 rpm. Then the temperature was set at 0° C. and after about 30 minutes of granulation the solid was separated on filter and dried at 60° C./1 hrs/N2.

### Example 8

### Preparation of Crystalline form F of Erlotinib Hydrochloride in Crystal16

25 mg of Erlotinib base+1 ml of 1,3-dioxalane (water content: 0.031%) were placed into a magnetic stirred glass-vial and dissolved. After that the temperature inside was adjusted at +60° C. and the solution was stirred for the duration of one hour. 5.25 μl of concentrated hydrochloric acid (44.1% w/v HCl; 1 eq) was added by a microsyringe, maintaining the stirring speed at 1100 rpm. A crystalline solid was appeared immediately after the addition. The temperature was kept at 60° C. for additional 10 minutes. Then the suspension was cooled down and after about 30 minutes of granulation at 0° C. the solid was separated on a filter and dried at 6° C./1 hrs/N2. Crystalline form F (17 mg; molar yield 62.2%) was obtained.

### Example 9

### Preparation of Crystalline Form F of Erlotinib Hydrochloride in Crystal16

25 mg of Erlotinib base+1 ml of 1,3-dioxalane (water content: 0.031%) were placed into a magnetic stirred glass-vial and dissolved. After that the temperature inside at +30° C. was adjusted and the solution was stirred for the duration of one hour. 5.25 μl of concentrated hydrochloric acid (44.1% w/v HCl; 1 eq) was added by a microsyringe, maintaining the stirring speed at 1100 rpm. A crystalline solid was appeared till about 10 seconds after the addition. The temperature was kept at 30° C. for additional 5 minutes. Then the suspension was cooled down and after about 30 minutes of granulation at 0° C. the solid was separated on a filter and dried at 6° C./1 hrs/N2. Crystalline form F (19.0 mg; molar yield 69.6%) was obtained.

### Example 10

### Preparation of Crystalline Form F of Erlotinib Hydrochloride

0.50 g of Erlotinib base+20 ml of 1,3-dioxalane (water content: 0.031%) were placed into a magnetic stirred bulb and dissolved. The temperature inside was adjusted at +30° C. and the solution was stirred for the duration of about one hour. 105 μl of concentrated hydrochloric acid (44.1% w/v HCl; 1 eq) was added via an electronic burette, maintaining the stirring speed at 700 rpm. A crystalline solid was appeared immediately after the addition. The temperature was kept at 30° C. for additional 10 minutes. Then the suspension was cooled down and after about 30 minutes of granulation at 0° C. the solid was separated on a filter and dried at 60° C./1 hrs/N2. Crystalline form F (0.52 g; molar yield 95%) was obtained.

### Example 11

### Preparation of Crystalline Form F Erlotinib Hydrochloride in Crystal16

30 mg of Erlotinib base+1 ml of 1,3-dioxalane (water content: 0.031%) were placed into a magnetic stirred glass-vial and dissolved. After that the temperature inside was adjusted at +30° C. and the solution was stirred for the duration of one hour. 6.30 μl of concentrated hydrochloric acid (44.1% w/v HCl; 1 eq) was added by a microsyringe, maintaining the stirring speed at 1100 rpm. A crystalline solid was appeared immediately after the addition. The temperature was kept at 30° C. for additional 10 minutes. Then the suspension was cooled down and after about 30 minutes of granulation at 0° C. the solid was separated on a filter and dried at 60° C./1 hrs/N2. Crystalline form F (20.5 mg; molar yield 75.1%) was obtained.

What is claimed is:

1. Crystalline erlotinib HCl characterized by data selected from the group consisting of: a powder X-ray diffraction (PXRD) pattern with peaks at about 5.9, 9.7, 11.7, 16.2, 21.7 and 23.3±0.2 degrees two-theta; a PXRD pattern depicted in FIG. 1; a PXRD pattern depicted in FIG. 2; a solid-state ¹³C NMR spectrum with signals at about 150.0, 136.1, 134.3 and 126.8±0.2 ppm; a solid-state ¹³C NMR spectrum having chemical shift differences between the signal exhibiting the lowest chemical shift and another in the chemical shift range of 100 to 180 ppm of about 48.4, 34.4, 32.6 and 25.2±0.1 ppm; a solid-state ¹³C NMR spectrum depicted in FIG. 4; and a solid-state ¹³C NMR spectrum depicted in FIG. 5, and combination thereof.

2. Crystalline Erlotinib HCl of claim 1, characterized by a powder XRD pattern with peaks at about 5.9, 9.7, 11.7, 16.2, 21.7, and 23.3±0.2 degrees 2-theta.

3. Crystalline Erlotinib HCl of claims 1 or 2, characterized by a powder XRD pattern as depicted in FIG. 1.

4. Crystalline Erlotinib HCl according to any one of claims 1-3, characterized by a powder XRD pattern as depicted in FIG. 2.

5. Crystalline Erlotinib HCl according to any one of claims 1-4, characterized by a solid-state ¹³C NMR spectrum with signals at about 150.0, 136.1, 134.3 and 126.8±0.2 ppm.

6. Crystalline Erlotinib HCl according to any one of claims 1-5, characterized by a solid-state ¹³C NMR spectrum having chemical shift differences between the signal exhibiting the lowest chemical shift and another in the chemical shift range of 100 to 180 ppm of about 48.4, 34.4, 32.6 and 25.2±0.1 ppm.

7. Crystalline Erlotinib HCl according to any one of claims 1-6, characterized by a solid-state ¹³C NMR spectrum depicted in FIG. 4.

8. Crystalline Erlotinib HCl according to any one of claims 1-7, characterized by a solid-state ¹³C NMR spectrum depicted in FIG. 5.

9. Crystalline Erlotinib HCl of claim 2, further characterized by a powder XRD pattern with peaks at about 11.3, 13.9, 19.1, 19.5, 22.5 and 24.5±0.2 degrees two-theta.

10. Crystalline Erlotinib HCl according to any one of claims 1-9, further characterized by a DSC thermogram having peaks at about 209° C. and 230° C.

11. Crystalline Erlotinib HCl according to any one of claims 1-10, further characterized by a DSC thermogram depicted in FIG. 3.

12. Crystalline Erlotinib HCl of claim 5, further characterized by a solid-state ¹³C NMR spectrum with signals at about 156.4, 154.4, 147.4 and 131.4±0.2 ppm.

13. The crystalline erlotinib HCl according to any one of claims 1-12, containing no more than about 15% by weight of crystalline Erlotinib HCl form characterized by PXRD having peaks at about 5.7, 9.8, 10.1, 10.3, 18.9, 19.5, 21.3, 24.2, 26.2 and 29.2±0.2 degrees 2-theta or crystalline Erlotinib HCl form characterized by PXRD having peaks at about 6.2, 7.8, 12.5, 13.4, 16.9 and 21.1 deg±0.2 degrees 2-theta.

14. The crystalline erlotinib hydrochloride HCl according to any one of claims 1-13, wherein the crystalline erlotinib hydrochloride HCl is anhydrous.

15. Crystalline erlotinib HCl characterized by data selected from the group consisting of: a powder XRD pattern having peaks at about 9.7, 11.2, and 21.1±0.2 degrees two-theta, and at least any 3 peaks selected from the list consisting of 5.6, 16.9, 24.0, 25.3 and 26.0±0.2 degrees 2-theta; a PXRD pattern depicted in FIG. 7; a PXRD pattern depicted in FIG. 8; a solid-state ¹³C NMR spectrum with signals at about 155.4, 148.6, 138.1, 129.4 and 102.3±0.2 ppm; a solid-state ¹³C NMR spectrum depicted in FIG. 10; and a solid-state ¹³C NMR spectrum depicted in FIG. 11, and combination thereof.

16. Crystalline Erlotinib HCl of claim 15, characterized by a powder XRD pattern having peaks at about 9.7, 11.2, and 21.1±0.2 degrees two-theta, and at least any 3 peaks selected from the list consisting of 5.6, 16.9, 24.0, 25.3 and 26.0±0.2 degrees 2-theta.

17. Crystalline Erlotinib HCl of claims 15 or 16, characterized by a PXRD pattern depicted in FIG. 7.

18. Crystalline Erlotinib HCl according to any one of claims 15-17, characterized by a

19. Crystalline Erlotinib HCl according to any one of claims 15-18, a solid-state ¹³C NMR spectrum with signals at about 155.4, 148.6, 138.1, 129.4 and 102.3±0.2 ppm.

20. Crystalline Erlotinib HCl according to any one of claims 15-19, a solid-state ¹³C NMR spectrum depicted in FIG. 10.

21. Crystalline Erlotinib HCl according to any one of claims 15-20, a solid-state ¹³C NMR spectrum depicted in FIG. 11.

22. The crystalline Erlotinib HCl according to any one of claims 15-21, further characterized by data selected from the group consisting of: a DSC thermogram having peaks at about 203° C. and 233° C.; a DSC thermogram depicted in FIG. 9, and combination thereof.

23. The crystalline Erlotinib HCl of claim 22, characterized by a DSC thermogram having peaks at about 203° C. and 233° C.

24. The crystalline Erlotinib HCl of claims 22 or 23, characterized by a DSC thermogram depicted in FIG. 9.

25. A formulation comprising at least one of the crystalline forms of Erlotinib HCl of any one of claims 1 or 15 and at least one pharmaceutically acceptable excipient.

26. A pharmaceutical composition comprising at least one of the crystalline forms of Erlotinib hydrochloride of any one of claims 1 or 15 prepared according to the processes of the present invention, and at least one pharmaceutically acceptable excipient.

27. Crystalline erlotinib HCl Form G containing no more than about 15% by weight of crystalline Erlotinib HCl Form A and no more than about 15% by weight of crystalline Erlotinib HCl Form B.

28. The crystalline erlotinib HCl of claim 27, wherein the crystalline erlotinib HCl contains a total of no more than about 15% by weight of crystalline Erlotinib HCl Form A and Form B.
